(19) United States
(12) Patent Application Publication
(10) Pub. No.: US 2025/0114075 A1
(43) Pub. Date: Apr. 10, 2025

(54) Ultrasound Imaging System with Enhanced User Interface

(71) Applicant: BFLY Operations, Inc, Burlington, MA (US)

(72) Inventor: Karl Thiele, St. Petersburg, FL (US)

(73) Assignee: BFLY Operations, Inc, Burlington, MA (US)

(21) Appl. No.: 18/909,514

(22) Filed: Oct. 8, 2024

Related U.S. Application Data
(60) Provisional application No. 63/588,742, filed on Oct. 8, 2023.
(60) Provisional application No. 63/588,738, filed on Oct. 8, 2023.

(51) Int. Cl. A61B 8/08; A61B 8/00

(52) U.S. Cl. CPC A61B 8/5223; A61B 8/08; A61B 8/4427; A61B 8/4488; A61B 8/461; A61B 8/467

(57) ABSTRACT

The systems and methods for capturing images using ultrasound and in particular, systems that provide an ultrasound probe having a two-dimensional (2D) array of ultrasound transducers elements. The systems leverage the 2D array of transducer elements to achieve beam steering over the generated ultrasound signals. The system applies beam steering to generate multiple ultrasound signals which are, in sequence, transmitted across the elevational dimension of the 2D transducer array and at different angles of orientation. Each transmission in the sequence can be treated as a slice of a final image (whether still or video) that can be presented on a display. The final image is generated by image processing the multiple different slices of images to join the images slices together into a composite image. The joined images may be presented on a display to the user. Optionally, the joined images may be presented as a video of images made by sweeping the ultrasound beam over an anatomical target.

### RELATED APPLICATIONS

This application claims the benefit under 35 U.S.C § 119 (e) of U.S. Application Ser. No. 63/588,742, filed Oct. 8, 2023, entitled “Ultrasound with Enhanced Imaging,” and U.S. Application Ser. No. 63/588,738, filed Oct. 8, 2023, entitled “Ultrasound Imaging System,” and all of which are hereby incorporated herein by reference in its entirety.

### FIELD

The systems and methods described herein relate to medical imaging devices and aspects of the technology described herein relate to collection of ultrasound data along different elevational steering angles to generate improved image viewing features for users.

### BACKGROUND OF THE INVENTION

Scientists and engineers have made remarkable advances with medical imaging technologies, including imaging probes that are portable, provide facile use and ready transport and produce clinically excellent images. Such devices allow doctors and other clinicians to use medical imaging for patients who are remote from hospitals and clinics with imaging equipment or who treat patients who are confined to home. As such, these portable systems provide patients access to the excellent care that advanced medical imaging can provide. Examples of such portable imaging systems include those described in U.S. Pat. No. 10,856,840 which is granted to the assignee hereof.

Although these systems work remarkably well, the demand for these systems extends to users who are less well trained than the typical sonographer. Today, sonographers are highly trained and often specialized in sonographic procedures such as imaging for abdominal, vascular, OBGYN, and echocardiography indications. These sonographers and other imaging professionals have learned excellent techniques for using and manipulating the ultrasound probe to collect images that are useful for the diagnostic effort or the procedure. Many of these techniques involve the careful maneuvering of the probe head across the habitus of the patient. These maneuvers distribute, orient and position the probe head in a way that collects a better, or more useful image for the treating clinician. Learning how to maneuver the ultrasound probe in this way is difficult. The ultrasound beam is, of course, invisible and this requires the sonographer to use indirect feedback as to the position and direction of the ultrasound beam. Often this feedback comes from watching the ultrasound image on a display. However, such images are often delayed in time from when the image was collected and they can be grainy and difficult for an unskilled user to decipher. In any case, getting visual feedback as the ultrasound image is being collected often requires the sonographer to develop a feel for how the image will appear on a display as the sonographers orients the probe head toward the anatomical target being imaged. The development of this feel can take quite a while to occur. Moreover, even when the skilled sonographers has developed the techniques for orienting the probe to capture images useful for the procedure, the technique developed by the stenographer may be somewhat individualistic and clinicians working with the images captured by different sonographers need to recognize that the collected image was taken using one type of technique versus another similar technique. For instance, some sonographers move the probe head across the habitus of the patient more quickly than other sonographers, and this can change certain characteristics of the collected images.

To extend the use of such instruments to a broader patient population who would benefit from the medical information provided by ultrasound devices, the devices need to become more facile to use. As such, there remains a need for systems that make the use of these sophisticated ultrasound imaging devices more facile for all the clinicians that can benefit from, and improve patient care, by the use of an ultrasound imaging system.

### SUMMARY OF THE INVENTION

The systems and methods described herein provide, among other things, systems for capturing images using ultrasound and in particular, systems that provide an ultrasound probe having a two-dimensional (2D) array of ultrasound transducers elements, typically capacitive micromachined ultrasound transducer (CMUT) elements that are arranged into an array. In one embodiment, the systems described herein leverage the 2D array of transducer elements to achieve beam steering over the generated ultrasound signals. The system applies beam steering to generate multiple ultrasound signals which are, in sequence, transmitted across the elevational dimension of the 2D transducer array and at different angles of orientation. Typically, the sequence of transmission is controlled so that the effect of the beam steering is to achieve an imaging procedure comparable to a sonographer rocking a probe head about a location on the habitus of the patient. Each transmission in the sequence can be treated as a slice of final image (whether still or video) that can be presented on a display. In some embodiments, the final image is generated by presenting the captured image slices as a video and in other embodiments the final image is generated by image processing the multiple different slices of images to join the images slices together into a composite image, or model. The joined images may be presented on a display to the user. Optionally, the joined images may be presented as a video of images that are presented in a cine video that presents the images in a way that simulates a sonographer sweeping the ultrasound beam over an anatomical target. As will be described herein, this can provide a useful image user interface which highlights anatomical features of the imaged anatomical target.

More particularly, in some aspects the systems and methods described herein include ultrasound systems having a handheld ultrasound imaging device including a flat two-dimensional (2D) array of micromachined ultrasound transducers (MUTs), and a processor configured to control the 2D array to take a series of ultrasound images along an elevational direction of the 2D array where each image is taken at a different angle relative to an axis parallel to the elevational direction of the array by beam steering ultrasonic signals produced by the MUTs. Typically the 2D array is rectangular and the elevational direction corresponds to the shorter dimension of the array. Further, the device includes a memory to store the series of ultrasound images as a series of ultrasound image data. Ultrasound image data includes information to generate ultrasound images on a display, such as the display on a smartphone or tablet, recorded in an electronic format suitable for storing in computer memory, processing by computer processing elements, and transmitting between electronic devices. Ultrasound image data can be stored in a computer memory as image data files. Beam steering refers to a technique for controlling the direction of travel of ultrasonic signals by controlling the transducers to propagate an ultrasonic wave in a direction other than one perpendicular to the surface of the array.

In some aspects, the image processor is configured to select an image from the series of ultrasound images data stored in memory and designates it as a key-frame. The key-frame may be an image in the series of images where the system determines the imaging target, or a pathology within the imaging target, is advantageously clear. The image processor is configured to display the key frame as a default still image, or as a central frame in a displayed video or cine. A cine is a short video which repeats by reversing back and forth through the frames of the video around a central frame. Depending on the application at hand any one of the display options may be preferable.

In some embodiments, the system includes a user interface to allow the user to select a different image from the series of ultrasound images and designate it as the key-frame. In some applications the user may determine that a frame other than the frame selected by the system is preferable as the key-frame. In order to view desired aspects of the imaging target, the user interface may allow a user to manually pause, fast-forward, and rewind a displayed video or cine. Depending on the application at hand, a user may determine that viewing the ultrasound image data as a still image, a video, or on a short loop is preferable.

The systems and methods described herein further provide, in one aspect, ultrasound imaging devices, whether portable, wearable, cart-based, PMUT, CMUT or otherwise, have controllers that have a real-time enhanced image display mode where the system may continuously sweep back and forth in the elevational dimension. Such a sweep feature may allow the user/clinician to survey the field faster and with more confidence to find areas of interest (e.g. b-lines, plaque, stones). As such, the imaging devices described herein with the enhanced image display includes a sweep function that may be understood, for purpose of illustration, as analogous to real-time b-mode, where the user slowly tilts the probe back and forth in the elevation direction. These systems and methods may further provide a user interface sweep function that will employ the beam steering and multiple images slices to create an display image for the user that continuously “rocks” the plane back and forth in the elevational dimension, with a periodicity of approximately one to two seconds. In typical embodiments, the sweep function is not automatically selecting the planes (in the example herein there are seventeen planes) in real time. One example intended use is to facilitate the observation of b-lines. B-lines tend to be better observed when the interrogating ultrasound plane is perpendicular to the pleural lining. Since it is difficult to know when one is perpendicular to this surface, an experienced user will rock the probe back and forth in elevation. The user interface sweep function described herein performs this function for such a user, helping to achieve an image capture suitable for a clinician to use.

To this end, in certain embodiments, the sweep function will present the multiple image slices collected during this simulated rocking process, and the result, as these are b-mode images, is that the b-mode image with the better or more perpendicular orientation will appear more brightly within the image formed from the images captured while rocking. This creates an image display that is a real time and varying composite of the b-mode slices collected during a sweep back and forth in the elevational dimension. This can cause the important parts of the image to jump out to the clinician as they brighten up as the more perpendicular orientation is achieved.

In some embodiments, the system is applied to image a target organ, where the sweep function will present the multiple image slices collected during this simulated rocking process. Due to typical variability in image quality, such as imaging angle or acoustic impedance, some imaging angles will generate images which more clearly display the target organ than other imaging angles. During operation of the system, the image processor is configured to analyze each image in the series of collected images to identify an image in the series of collected images where the target organ is displayed more clearly than in other images in the series of images. The system may then designate that image as the key frame, this can cause the target organ to jump out to the clinician as it becomes clearer as the more advantageous imaging angle is achieved.

In some embodiments, the system is applied to an anatomical structure within the habitus of the patient where a pathology is visible in an ultrasound image. The sweep function will present the multiple image slices collected during this simulated rocking process. Due to typical variability in image quality, such as imaging angle or acoustic impedance, some imaging angles will generate images which more clearly display the pathology within the anatomical structure. During operation of the system, the image processor is configured to analyze each image in the series of collected images to identify an image which more clearly displays the pathology compared to other images in the series of images. The processor may designate the clearer image as the key frame, this can cause the pathology to jump out to the clinician as it becomes clearer as the more advantageous imaging angle is achieved.

### BRIEF DESCRIPTION OF THE DRAWINGS

The systems and methods described herein are set forth herein and, for purpose of explanation, several embodiments are set forth in the following figures.

FIG. 1 depicts one embodiment of the systems described herein;

FIG. 2 depicts pictorially a system having a controller for controlling imaging of a probe and for operating the probe in a sweep configuration;

FIG. 3 is a block diagram showing a probe, such as a probe of FIG. 1, and how in the sweep mode the planes are swept in the elevational dimension.

FIG. 4 is an example image from a video generated of real-time B-Mode of a lung (between the intercostal spaces): in this video, showing radial lines that streak down from 3 to 12 cms, with the scale on the right side given cms. B-lines are indicative for fluid in the lung, and can be used to diagnose Congestive Heart Failure, Pneumonia, Covid and more;

FIGS. 5A and 5B depict a real-time image capture with the methods described herein, where the plane rocks back and forth, and depict in stills of a video from the lung imaged in FIG. 4, “b-lines” (radial streaks from the “pleura” located at the 3 cm depth). Although FIGS. 5A and 5B are still images it will be understood by one of ordinary skill in the art that this is a moving image that moves back and forth through the sequence of collected image slices;

FIG. 6 depicts three side-by-side examples of images captured with the methods described herein, the first being the carotid artery, the next being the vertebral artery and the third being the lung; although FIG. 6 presents the moving images as still images it is to be understood by those of skill in the art that all three are moving images made from a composite of slices taken by the transducer sweeping about 20 degrees about the center angle;

FIG. 7 is an example image of a static B-mode Image of a transplant right kidney;

FIGS. 8A and 8B depict a cine generated using the methods described herein and shows a kidney from upper to lower pole and in this example hydro was identified in the upper and mid poles; although FIGS. 8A and 8B present a cine loop as two individual images, it will be understood that this is a video that moves back and forth through the sequence of collected image slices or a 3D model made from those image slices;

FIG. 9 depicts a process wherein the user can select a keyframe; and

FIG. 10 depicts a process wherein the user can activate the sweep feature.

### DETAILED DESCRIPTION

In the following description, numerous details are set forth for purpose of explanation. However, one of ordinary skill in the art will realize that the embodiments described herein may be practiced without the use of these specific details. Further, for clarity, well-known structures and devices are shown in block diagram form to not obscure the description with unnecessary detail.

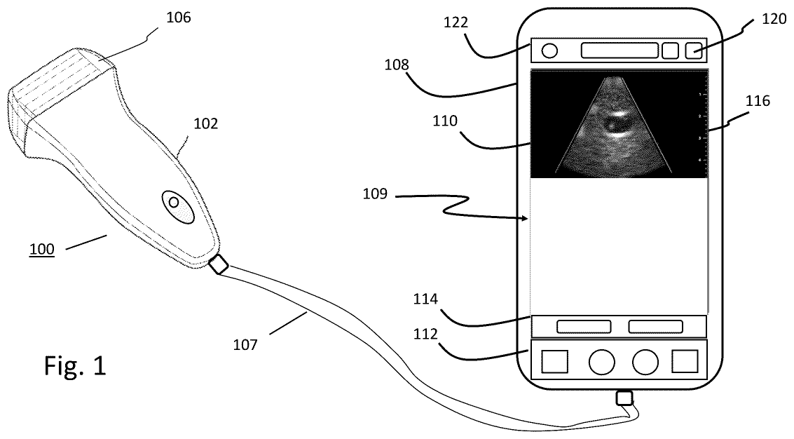

In one embodiment, the systems and methods described herein include, among other things, the system 100 depicted in FIG. 1. The system 100 depicted in FIG. 1 is a handheld ultrasound probe of the type that can connect to a smartphone or a tablet to display ultrasound images, including still images and video, to a clinician. Although the systems and methods described herein may be used with cart-based ultrasound system and other ultrasound imaging systems, for purposes of illustration, the systems and methods described herein will be described with reference to ultrasound handheld probe systems such as the system 100. These handheld systems are more portable and less expensive than traditional cart-based ultrasound systems and as such provide the benefits of ultrasound imaging to a larger population of patients. One such portable handheld system is the iQ series of ultrasound probe systems manufactured and sold by the Butterfly Network Company of Burlington Massachusetts.

As will be described herein, the portable system 100 of FIG. 1 includes a user interface feature that allows the user to place the probe 100 (in this case the probe head 106) against the patient and sweep through a sequence of imaging processes that collect a sequence on image slices. The system 100 will join the image slices to create a 3D model of the anatomical target imaged with probe. The system 100 will allow the user to view that 3D model of the anatomical target from alternate perspectives. Additionally, the image slices may be presented as a sequence of images that are presented in a video format and allow the user to see the different image slices in sequence. Typically, but optionally, the system allows the user to select an image frame from the presented video to act as a key frame, typically selecting the image frame that most clearly presents the anatomical feature of interest.

To this end, and to illustrate such a system in more detail, FIG. 1 depicts the system 100 that includes a probe 102 and a handheld device 108. The probe 102 has a transducer head 106. The probe 102 is coupled by a cable 107 to a handheld device 108, depicted in FIG. 1 as a mobile phone. The depicted handheld device 108 is executing an application that collects data, including image data, from the probe 102. The application may display a live and moving image within the image window 110 and may display within software UI windows, such as the software UI window 112, one or more controls for operating the probe 102, such as a control for enabling sweep operation that will direct the probe 108 to take a sequence of image slices across the elevational dimension of the transducer array and to join the image slices into a single model that can provide a 3D model of the anatomical target. Additionally, the user interface controls in window 112 may allow the user to present the image slices on the display of the handheld, such as depicted by image 116, to show the sequence of image slices collected by the transducer head. In FIG. 1 the system is a handheld probe, but as noted above this sweep mode can easily be extended to a cart-based system.

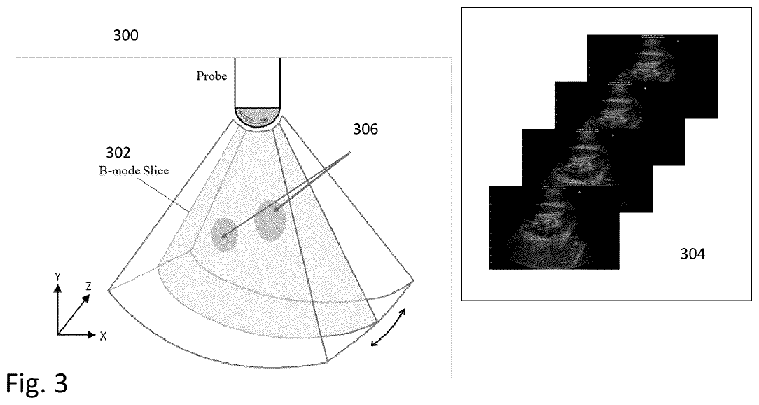

The probe 102, in this example embodiment, is an ultrasound probe of the type disclosed in U.S. Pat. Nos. 10,856,840 and 11,167,565 both assigned to the assignee hereof. The probe 102 is a handheld ultrasonic imaging probe that can be used by the clinician to image a patient and collect medical images useful in the clinical process of diagnosing and treating the patient. The probe 102 has a transducer head 106 that the clinician may place against the tissue of the patient, such as by placing the transducer head 106 in contact with the patient's chest proximate to the heart or lungs of the patient or proximate the kidneys, or wherever the clinician wishes to image. In typical operation, the clinician uses a UI control button as a UI by which the clinician may activate various functions such as image capture operations that cause the application 109 executing on the handheld device 108 to store one of the images, such as depicted in FIG. 3, generated by the transducer head 106. That application 109 may render the captured images in the image window 110 for the clinician to view. In the sweep mode the images (the image slices) may be presented as if the probe were being physically rocked back and forth, rather than looped through the images.

The UI window 112 may provide the clinician with a series of optional user interface controls that the clinician may use to operate the application 109 executing on the handheld device 108 to change how the captured image is rendered, store the image, mark the image, and perform other types of operations useful during the tomographic procedure.

During a tomographic procedure or other imaging process, the clinician adjusts the position and angle of the probe 102 until an image of interest appears in the image window 110. In some embodiments, the clinician may activate the UI control button to capture images to study, or activate various functions such as the sweep/slice mode.

In the embodiment depicted in FIG. 1, the handheld device 108 is a programmable device that runs the application 109 that, for example, performs the image display functions, user interface functions, such as allowing the clinician to select presets and capture images from the image stream, and configures the system 100 with any selected preset parameters. In this embodiment, the handheld device 108 may be a smart phone, a tablet, or any other suitable handheld device capable of running application programs and of supporting a data connection to the probe 102. In the depicted embodiment the handheld device 108 couples to the probe 102 by way of the cable 107. However, in alternative embodiments, the handheld device 108 and the probe 102 may have a wireless connection of the type suitable for transferring data and control signals between the two devices. In one example, the wireless connection may be the Bluetooth protocol (IEEE 802.15.1), ultra-wideband (UWB, over IEEE 802.15.3), ZigBee (over IEEE 802.15.4), or Wi-Fi (over IEEE 802.11) connection or a connection using some other protocol for short-range wireless communications, preferably with low power consumption. However, any suitable wireless technology may be used including those that work with narrow bands, employ optical communication, or which use some other suitable technique for exchanging information between the probe 102 and the handheld device 108.

In the embodiment depicted in FIG. 1 the transducer head 106 includes an array of ultrasonic transducer elements. The array of ultrasonic transducers may be a 2D array of MEMs transducer devices, such as an array of capacitive micro-machined ultrasonic transducers (CMUTs) or an array of piezoelectric micromechanical ultrasonic transducers (PMUTs), that are capable of generating ultrasonic waves, including beamformed ultrasonic waves and detecting ultrasonic waves as they return from the patient. In one embodiment, the depicted transducer head 106 includes thousands of transducer elements that operate in coordinated action to create the ultrasound beam used for the image collection. In one example, the transducer head 106 includes a two-dimensional array of thousands of transducer elements organized into a 2D array and formed on a semiconductor die or chip. The die or chip may, in certain embodiments, further contain on-chip processing circuitry including more than one thousand analog-to-digital converters and amplifiers. Embodiments of transducers formed on a semiconductor die or chip are shown in more detail in U.S. Pat. No. 9,067,779 and in US application US2019/0275561. Embodiments of on-chip processing circuitry are shown in more detail in U.S. Pat. No. 9,521,991. In other embodiments, the transducer head may use PMUTs and the A/D converters and amplifiers may be on separate chips or dies and the chips and dies may be mounted on a circuit board or boards.

In operation, the transducer head 106 detects ultrasonic waves returning from the patient and these waves may be processed by processing circuitry formed on the same chip as the transducers, a signal processor, a CPU, an FPGA, or any suitable type of processing device, or any combination thereof, which may process the returned ultrasound waves to construct image data. That image data may be used by the application 109 running on the handheld device 108 to create images for the clinician.

In the depicted optional embodiment, the executing application 109 may include an image processor. The image processor is, among other things, configured to analyze the ultrasound image data stored in the system's memory (on the handheld or on the cloud for instance) and a cine with key frame and optionally to join the image slices together into a 3D model that can be stored in a data memory and presented in a display. The presentation of the joined image can be as the constructed images, including video images, such as the ultrasound image cine 116, on the image window 110 so that the clinician can see images of the patient as those images are being generated by the probe 102 or when the physician wishes to review the captured images. In the depicted embodiment, the application 109 also provides a UI window 112 that has a series of software control buttons, sometime called widgets, that the clinician may use for controlling the operation of the probe 102. These controls allow the clinician to change how images, such as image cine 116 are rendered, captured, and displayed on the handheld device 108. Although, the embodiment of FIG. 1 is described with a system that creates a 3D model, it will be understood that in other embodiments the image slice data are stored as data files in a memory and the cine presented to the user is generated by a image processor and video processor that present the image slices to the user in sequence to create a cine that moves forward and reverse through the image slices or a portion of the image slices.

In the embodiment of FIG. 1, the application 109 further has a menu 114 that depicts a preset selected by the clinician and an indication of the view selected for the clinician.

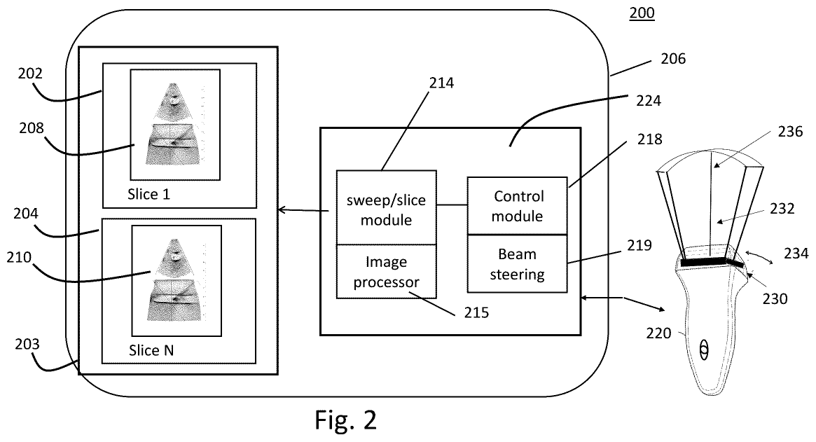

In one embodiment, the application 109 implements a sweep feature of the type depicted pictorially in FIG. 3. FIG. 2 depicts pictorially and in more detail a system 200 that has a handheld device 206 (such as a smartphone) executing an application 224 that may configure the system 200 to carry out the sweep/slice feature described herein. As will be described in more detail below, the user, typically the clinician taking the image, can employ the user interface of the system, such as by selecting a widget from the software UI window 112 that will put the probe and system into a mode to take multiple image slices in a sequence of image slices that sweep across the elevational dimension of the transducer array of the probe. Typically, in this sweep/slice mode the clinician will place the probe on the habitus of the patient at a location the clinician deems correct for imaging the anatomical target of interest, such as the patient's kidney. The clinician can place the probe so that the probe is essentially “flat” against the habitus of the patient. In this orientation the transducer array of the probe is such that an axis extending outward from the array and perpendicular to the array will extend into the patient and toward the anatomical image target of interest. In this sweep/slice mode the clinician will hold the probe steady so that the axis continues to extend toward the anatomical target of interest. The sweep/slice mode will operate the probe to, during a time period of 1-5 seconds for example, take a series of still images, each at a different, and typically contiguously progressing angle relative to the axis extending from the transducer array. In one example, the sweep/slice moder will capture about 45 still images, each at a different elevational angle and collectively sweeping through an arc of about 20 degrees to either side of the axis extending outward from the face of the transducer array.

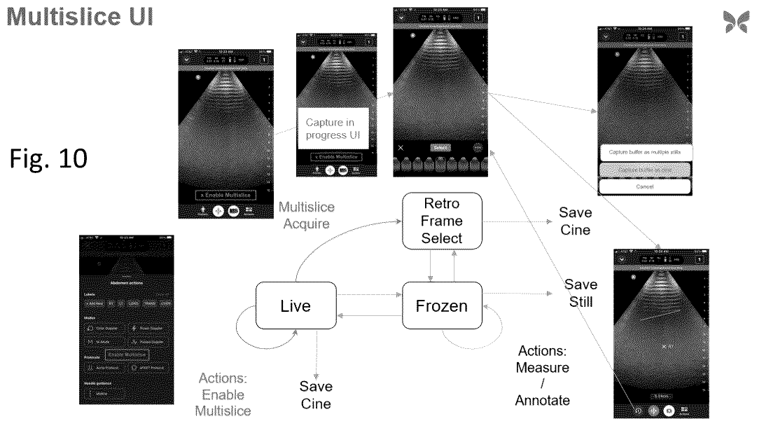

As described below, in particular with reference to FIG. 10, the user interface may allow the clinician to select an auto fan mode that will sequentially present a portion of the images to simulate viewing the anatomical area as if the clinician were manually fanning or rocking the probe during imaging. Additionally and optionally, the system can join the different still image slices together to build a 3D model of the area imaged and provide the clinician with an interface tool to allow the clinician to view the model from different perspectives.

To this end and to implement these functions such as sweep/slice mode, auto fan, and building the 3D model, the system 200 includes an application 224 that typically is a computer program executing on a processor built within the circuit board of the handheld device 206. For clarity and ease of illustration, the application 224 is depicted as a functional block diagram and as an application running on the handheld. In alternate embodiments, the application can be run, at least in part, on the cloud and data stored on the cloud.

The functional blocks of application 224 include a control module 218, a beam steering module 219, a sweep/slice module 214 and an image processor module 215. The handheld device 206 also includes a video module 203. The video module 203 can handle video tasks, such as capturing streams of video data generated by the probe 220 and rendering the video on the display of the handheld device 206. In one example, the video module 203 in cooperation with the image processor 215 converts the collected images 208 and 210 generated by sweep/slice mode module 214 that in one embodiment will allow the user to implement the auto fan presentation that simulates manually continuously “rocking” the image plane back and forth in the elevational dimension, with a periodicity of for example approximately 1 to 2 seconds, 3 to 5 seconds, or whatever time period is suited for the application, such as kidney imaging. The video module and the image processor will thereby generate a forward and reversing cine centered on a key frame. In particular, in one embodiment the image processor is configured to analyze the ultrasound image data files stored in memory. The image processor will designate an image slice (whether an actual image slice or one simulated from taking a slice form a 3D model) from the series of ultrasound images as a key frame. The video module and image processor can then, in some embodiments, select several image slices that occur in the sequence of images before the keyframe and several images slices occurring in the sequence of images after the keyframe and generate a cine that will play on the display moving forward and reverse through the selected subset of image slices and centered on the key frame. As shown in FIG. 2, the video module 203 includes image memory 202 and 204 that can store respective image data for each slice, such as the depicted image slices 210 and 208. The image slice image data 208 and 210 is the data collected by the beam-steer-controlled array 230. The number of image slices taken may differ depending upon the simulated rocking process (how much of a rocking angle and how long the rocking takes) and the periodicity may vary. For example, it may vary depending upon the preset being used, the anatomical organ being imaged, the depth of the imaging and other parameters. Additionally, the user may have some control over the periodicity and may change the period based on watching the image and selecting a period that causes features in the live image from sweep mode becoming more clear to the user within the display. In FIG. 2 only two image slices are shown, but it will be understood that the sweep/slice module 214 typically takes more image slices, and for example taking 40 to 50 image slices during a sweep/slice operation is common. The cine may be made from a portion of the 40-50 images slices, such as 4-10 or 6-18 image slices, or the number of image slices suited for generating a cine useful for the auto-fan user interface feature.

FIG. 2. further depicts that the handheld device 220 includes a 2-dimensional (2D) array of ultrasound transducers 230. FIG. 2 depicts the array 230 using perforated lines as the array 230 is typically encompassed and covered by an acoustically transparent housing made of a biocompatible material that is suitable for contacting the habitus of a patient. FIG. 2 further depicts that the transducer array 230 is operated by the control module 218 and the beam steering module 219 to generate an ultrasound signal that is angled relative to the face of the transducer array 230. In one embodiment, the control module 218 generates operating parameters that can be provided to the 2D array 230 to control operation of the array 230 and typically to separately control each transducer in the 2D array 230. Parameters can include frequency of operation, duration of operation, timing, and therefore relative phase of operation, and other parameters relevant to operation of the array 230. In one embodiment, the array 230 includes or is connected to an ASIC or microcontroller that can collect operating parameters from the control module and load them into the ASIC in a manner that will allow the parameters to control operation of the individual elements of the transducer array 230. The beam steering module 219 in one embodiment generates a beam steering parameter for the control module 218 to deliver, with other operating parameters, to the transducer array 230. The beam steering module 219 can generate the parameters needed to sweep the ultrasound beam 232 across the elevational angle 234 of the transducer array 230, and take the number of image slices suited to the application, such as 40-50 image slices across for example a 20-30 degree arc about an axis 236 perpendicular to the face of the array 230.

FIG. 2 depicts one such ultrasound signal, 232, which represents an ultrasound signal employed to generate one image slice. In this embodiment, the beam steering module 219 will direct the handheld device 220 to operate the 2D transducer array 230 to generate multiple image slices, such as 10 to 20 image slices, 20 to 30 image slices, 40 to 50 image slices or which number of slices is the suitable number of image slices for the application at hand. In any case, it can be seen from FIG. 2 that the image slices are to be each directed along a different elevational angle by using the beam steering module 219 to control, typically, a phase shift that is applied to the ultrasound signals generated independently by respective ones of the transducer elements within the 2D array of transducer elements 230. Techniques, for phase shifting an array of transducers for transmitting and receiving ultrasound signals are known in the art, and any suitable technique for such beam steering may be employed without departing from the scope of the invention. Such techniques are disclosed in for example, Kinsler et al.; Fundamentals of Acoustics; John Wiley and Sons, pages 188-204 (2000); and Shi-Chang Wooh, Yijun Shi; Optimum beam steering of linear phased arrays, Wave Motion, Volume 29, Issue 3, 1999, Pages 245-265.

Optionally, the systems and methods may use beam steering and adjust the aperture of the array 230 during sweeps across the elevational arc 234. To this end, the beam forming module 219 may configure the array 230 for successive iterations of transmitting and receiving ultrasound waves. Each set of ultrasound data collected from the successive iterations of transmitting and receiving ultrasound waves may be focused at different elevational steering angles using beamforming. The different elevational steering angles may be measured relative to the axis 236 extending outward and perpendicular to the face of the ultrasound transducer array 230. The beamforming process implemented by module 219 may include applying different timing/phase delays to the transmitted and received ultrasound waves/data from different portions of the ultrasound transducer array 230 such that there are different delays for different elevational rows, where a row refers to the transducer elements spaced along an line extending along an azimuthal direction of the ultrasound transducer array. Similar delays may be applied to all elements in a row, or each element in a row, or each element in the array 230, may have a separately determined delay. The technique used will depend on the application. Optionally, and as disclosed in U.S. Pat. No. 11,167,565 assigned to the assignee hereof, the aperture of the array 230 may vary depending, in part, on the elevational steering angle to address differences in signal-to-noise ratio for the ultrasound data collected when the steering angle for the data is different from a zero elevational steering angle. In this embodiment, the signal-to-noise ratio at more extreme elevational steering angles may be improve, typically increased, by transmitting ultrasound waves from and receiving ultrasound waves with a larger elevational aperture of the ultrasound transducer array 230 (i.e., by using more elevational rows). In this embodiment, the beamforming module 219 may vary the elevational aperture during an elevational sweep as a function of elevational steering angle. In particular, the elevational aperture may be increased with more extreme elevational steering angles. In some embodiments, the number of elevational rows used at different iterations of transmitting and receiving ultrasound waves during the sweep may vary for example, between approximately 2 and 64.

In operation, the user may hold the probe 220 in place against the habitus of the patient and the beam steering module 219 will generate, in response to a user interface command entered by the user, ultrasound signals, such as the depicted ultrasound signal 232, each of which is shifted along the elevational direction 234 to sweep the image slices 232 in an arc 234 across and over the elevational direction of the transducer array 230. This coordination of the sweep/slice module 214 and the control module 218 and the beam steering module 219 provide automated sequential ultrasound capture mode on the handheld device 220, by having the control module 218 send the appropriate parameters to the transducers elements of the array 230 as directed by the beam steering module 219 to thereby automatically steer the beam 232 to scan an organ and capture multiple ultrasound image slices at one time and across a wide angle. The sweep/slice imaging mode is designed to make it easier and faster to acquire excellent images without skilled maneuvering. These image files 208 and 210 generated and stored in memory 203 can either be immediately read and measured at the bedside by skilled scanners or, for those less experienced, can be sent to a specialist for further review, similar to the workflow of a CT or MRI. Additionally, as noted above, the system 200 provides a tool, for example for use with lung imaging, that allows users to capture and view real-time back-and-forth virtual fanning, making it easier to visualize for example A-lines and other lung pathology.

The development of applications such as the application 206 with the sweep module 218, the beam steering module 219 and the video module 203 that execute on a handheld device such as a smartphone or tablet and that carry out the depicted functions of the application 206 is well known to those of skill in the art. Techniques for developing such applications are set out in for example, Alebicto et al., Mastering iOS 14 Programming: Build professional-grade iOS 14 applications with Swift 5.3 and Xcode 12.4, 4th Four ed.; Packt Publishing Ltd (2021).

In optional embodiments, the systems described herein may be responsive to the type of imaging operation that the clinician is undertaking. In certain imaging devices, such as those described in U.S. Pat. No. 10,709,415 assigned to the assignee hereof, the probe 102 may be placed into a preset, such as one for Kidney. Table 1 below lists certain example presets.

TABLE 1

Preset
Abdomen
Abdomen Deep
Aorta & Gallbladder
Bladder
Cardiac
Cardiac Deep
Coherence Imaging
FAST
Lung
MSK-Soft Tissue
Musculoskeletal
Nerve
OB 1/GYN
OB 2/3
Ophthalmic
Pediatric Abdomen
Pediatric Cardiac
Pediatric Lung
Small Organ
Vascular: Access
Vascular: Carotid
Vascular: Deep Vein

Each preset is a mode adapted for a particular type of imaging study. Presets may help with imaging studies and may be employed within systems that have the sweep feature described herein.

FIG. 3 shows in more detail how the probe sweeps the ultrasound beam across an anatomical area. The probe 308 is positioned on the patient and collects B mode images slices that can be used by the image processor 215 to generate a forward and reversing cine centered on a key frame and optionally to construct a 3D model of the anatomical area being imaged. The probe 308, as described above, may be operated in a way that beam steers across the elevational direction to effectively rock back and forth while taking an image periodically such as once every one or two seconds. In one embodiment, the probe travels through about 20 degrees of motion typically ±10 degrees above the center angle (that is relative to an axis extending outward and perpendicular from the face of the transducer array of probe 308). In the example depicted in FIG. 3, the probe 308 is collecting B Mode image slices. The B mode image slices collected during this rocking include B mode image slices such as depicted B mode image slice 302. These B mode image slices can be uploaded to a memory such as the memory 304 that will store the images as image data.

The sweep/slice module 214 depicted in FIG. 2 can collect the images and in cooperation with the video module 203 present them to the user in response to an auto fan mode selection made by the user through the user interface 112. In auto fan mode a live image is presented to the user on the handheld device. In one embodiment, the sweep/slice module 214 in cooperation with the video module 203 will present the images as if they are simulating a rocking maneuver of a trained sonographer, that is by passing through the images in one direction and then passing through them in the other direction. In other words, the images are displayed sequentially from a first angle to the furthest angle and then from that furthest angle back to that first angle. This is different than looping through the images. The bounced image or yo-yo image are brightness mode images and therefore features of interest to the clinician will from time to time appear more brightly within some of the slices. By presenting the sequence of images as a cine moving back and forth through the image slices, certain bright features will appear naturally and the display will have them appear more prominently and more easily observable by the clinician. This provides a user interface feature that can cause certain features of the anatomy to appear as an enhanced image on the screen such that features of interest are highlighted more brightly than others.

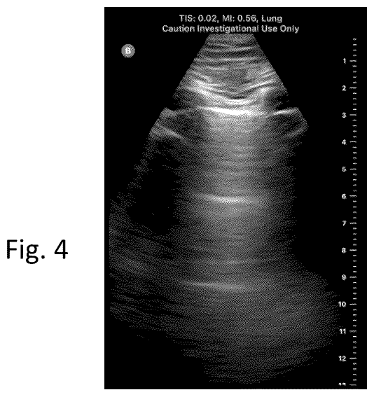

FIGS. 4-6 depict actual example images and videos of the type that are captured and presented to users with the systems and methods described herein. Specifically, FIG. 4 presents an example of a single image slice from an actual live image taken over a time period of a few seconds, during which a system of the type described herein took multiple image slices. The image of FIG. 4 is a single image slice showing a B mode image. In particular, FIG. 4 is a real-time B-Mode of a Lung (between the intercostal spaces). As a real-time B-mode image, it is a live image much like a video or gif. In this video shown by a single image in FIG. 4, one can see B-lines (radial lines that streak down from 3 to 12 cms). B-lines may be indicative of fluid in the lung, and can be used to diagnose Congestive Heart Failure, Pneumonia, Covid and other indications. Such images provide some information to a clinician but certain features may be more difficult to see because of the particular slice and the orientation of that real time live image slice relative to features of interest to the clinician.

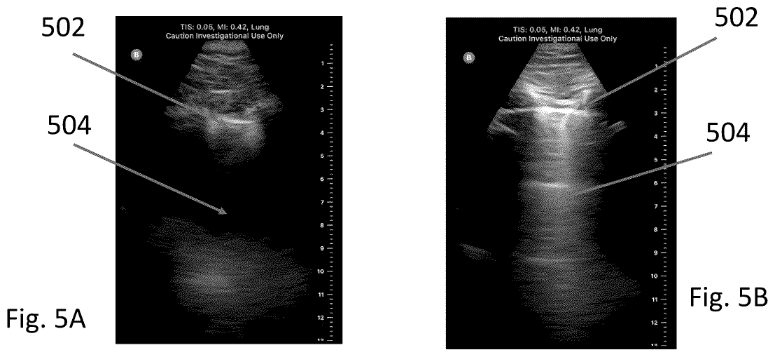

In contrast FIGS. 5A and 5B depict a live image that is being “rocked” (that is a cine running forward and back through the image slice data is simulating a rocking motion of the probe). The “rocked” images are displayed during an auto fan user interface operation to show the image slices across a defined range of angles collected by the sweep/slice module. It will be noted, that for the purpose of preparing this disclosure, video and live images may not be presented fully, and FIGS. 5A and 5B merely represent such a live or moving image by presenting two screenshots of that moving image. But it will be understood by those of skill in the art that this image is actually a live or moving image; one that is generated by the sweep/slice module passing through and presented by video module 203 on the display, in sequence, the different images collected by the probe 308 while the probe 308 takes images as it steers the ultrasound beam through different elevational angles to simulate rocking back and forth between a first angle and a second angle.

In any case, FIGS. 5A and 5B depict ultrasound images collected from the same lung as at the same time as shown in FIG. 4, but in sweep/slice mode. One can see that in sweep/slice mode the image will change and different features such as the depicted feature 502 and the depicted feature 504 will appear as the image is presented as being rocked from the first angle to the second angle and back again. This allows certain features of interest to the clinician to appear more clearly and be more easily recognized by the clinician.

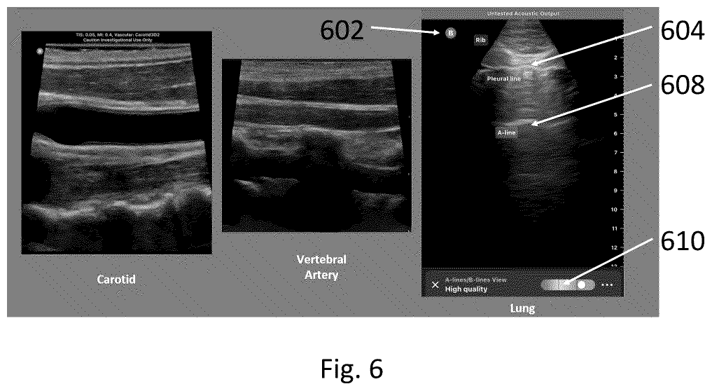

FIG. 6 depicts three side by side examples of images captured with sweep/slice mode, the first being the carotid artery, the next being the vertebral artery and the third being the lung. Again, although FIG. 6 presents the sweep/slice mode moving images as still images it is to be understood by those of skill in the art that all three are moving images, like gifs, or bouncing live images of an smartphone display, and made from a composite of slices taken by simulated rocking of the transducer back and forth by about 20 degrees about the center angle. It will be understood to those of skill in the art that the simulated rocking may be carried out by software directing the beam by controlling the transducers in the ultrasound head to achieve a selected angle.

Further, FIG. 6 shows an image on the right hand side of FIG. 6 of a sweep mode image that presents the B mode images being rocked back and forth to provide an enhanced display to the clinician. Additionally, this right-hand image is further enhanced to display annotations and includes indications of where the patient's ribs are located 620, the pleural line 604 of the patient and an A line 608 within the lung of the patient as well. On the lower right-hand side an widget 610, in this example an oval shape with a circle in it, indicates where within the angular sweep of the current image occurs. In operation the circle within that oval section widget 610 can move back and forth indicating that the live image is formed from a composite of the slices taken by rocking the transducer back and forth during imaging. The location of the circle within the oval indicate the relative angle of the slice currently being displayed.

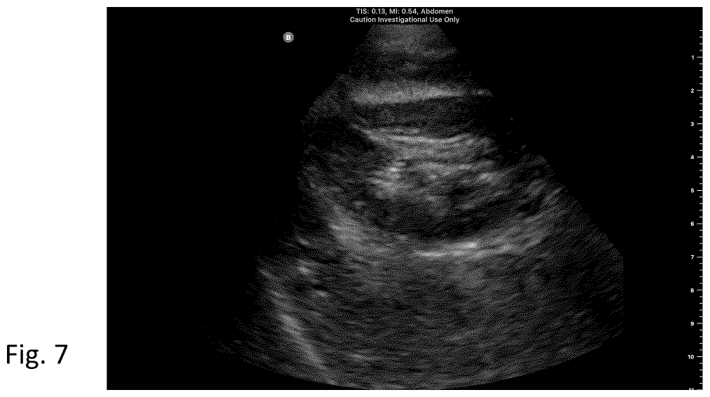

Returning to FIG. 3, it is noted that FIG. 3 shows the probe 308 taking multiple B-mode slices with one B-mode slice depicted individually as the slice 302 and the memory 304 storing actual examples of B mode image slices. The B-mode image slices can be collected and stored by the system 100 in memory such as in cloud storage memory and this is shown by the cloud storage 304 which stores the multislice images. Each individual slice may show some information about the anatomical feature being imaged, but collectively a cine may be run as a back and forth traveling sweep image that is looped and this can provide more comprehensive review of the anatomical organ being imaged. For example, in the actual examples of FIGS. 3, 7 and 8A and 8B, a kidney is being imaged. The probe can take B-mode slices 302 from one pole to the other pole. Any particular slice, such as that depicted in FIG. 7, may fail to show the collection of water within the kidney. However, operating the system so that the image presentation is in cine mode using the multislice images to generate a cine, renders the presence of hydro/water within the kidney more apparent and more easily viewable by the clinician. Thus, the B slice image depicted in FIG. 7, may not present the collection of hydro/water within the kidney in a way that is readily apparent to all clinicians.

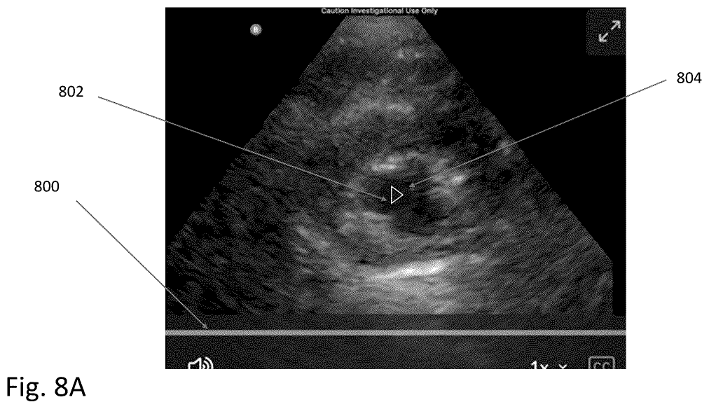

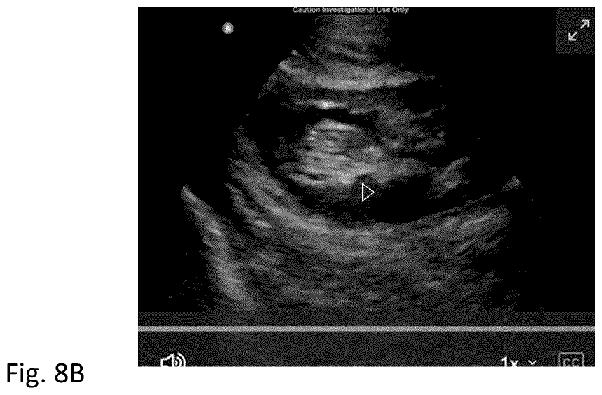

In contrast FIGS. 8A and 8B, (which are only two screenshots of the cine developed and presented to the user by the systems and methods described herein) present a cine loop of multislice images moving from the superior pole to the inferior pole of the kidney. That FIGS. 8A and 8B present a video cine is apparent from the time bar 800 and the play arrow 802. It further can be seen that the image in FIG. 8A includes a feature 804 that is not readily apparent in FIG. 7. The feature may grow more apparent as the cine loops, such as in FIG. 8B. Even though FIG. 7 is a B slice image of the same kidney, it is FIGS. 8A and 8B that actually to present more information, or at least different information, to the clinician. With this image and visual context provided by the cine, the clinician is much more able to identify the presence of hydro within the kidney.

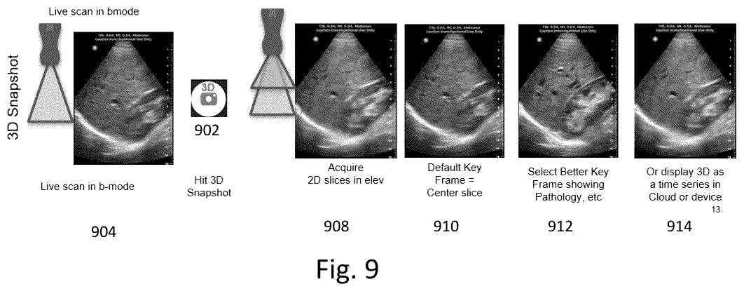

FIG. 9 depicts an example of the system selecting a preferential key frame, and the user interface allowing the user to pick a different key frame. The system scans the image as multiple slices by beam steering. As discussed above, the image slices 904 can be stored as image slice data files 908 stored in memory, and optionally combined into a 3D model. In either case, the system selects a default key frame 910 from the set of ultrasound image data files 908 and presents the ultrasound image data files 908 to the user as either a video of the individual slices or after they have been combined into a model. They system may alternately determine that a different slice 912 displays a preferential view of an imaging target, in this example a pathology, and designate that slice as the key frame. The system determines that a different slice displays a preferential view, depending on the application at hand, if the slice displays an imaging target is more clear, a pathology is more visible, or if b-lines are more prominent in the selected image 912 relative to other images data files 908.

The user interface allows the user to select a different frame as the key frame if the user determines that a different slice is preferable. In a typical case, the user will do a b-mode scan 904 using the beam steering process discussed above. Then, will use the user interface to start a cine which present the slices back and forth, as if the user were rocking the probe across the habitus of the patient. In one example, the scan has collected 46 image slices. It is often the case, that only a small number of those image slices, perhaps 4 or 6, present in good detail the anatomical feature of interest for example a pathology or b-lines. In operation, the user can select the snapshot button 902 at a point during the cine where the pathology gives the user's preferential view. The selected slice is replaced as the key frame.

In particular, FIG. 9 depicts a user interface having a snapshot button 902 which actives key frame selection from a series of image slices 904 collected and stored ultrasound image data files 908 stored in memory. The system will select, from the ultrasound image data files 908, a default key frame 910, in the presented example the center slice, and display a cine rocking back and forth around the key frame 910. The system may select an alternate frame 912 showing a preferential view of an imaging target and designate that slice as the key frame. The user may then use snapshot button 902 to select a different frame from the presented cine and designate that as the key frame.

FIG. 10 depicts the multi slice user interface. In the illustration of FIG. 10, different screenshots of the user interface are presented. FIG. 10 shows the actions of enabling the multi slice, the multi slice acquisition, the fact that the capture is in progress can display on the user interface, if a key frame can be selected by default or the user can select a keyframe, and the user has the option to store slice data as captured buffered data, as multiple stills in the capture bugger or as a cine, once saved the information can be presented as stills or as a cine as also depicted in FIG. 10. Optionally, measurements may be taken and annotations may be added to the images that are required.

The systems and methods described herein reference circuits, processors, memory, CPUs and other devices, and those of skill in the art will understand that these embodiments are examples, and the actual implementation of these circuits may be carried out as software modules running on microprocessor devices and may comprise firmware, software, hardware, or any combination thereof that is configured to perform as the systems and processes described herein.

Further, some embodiments may also be implemented by the preparation of application-specific integrated circuits (ASICs) or by interconnecting an appropriate network of conventional component circuits. To illustrate this interchangeability of hardware and software, various illustrative components, blocks, modules, circuits, and steps have been described herein generally in terms of their functionality. Whether such functionality is implemented as hardware or software depends upon the application and design constraints imposed on the overall system. Skilled artisans may implement the described functionality in varying ways for each particular application, but such implementation decisions should not be interpreted as causing a departure from the embodiments described herein.

Accordingly, it will be understood that the invention is not to be limited to the embodiments disclosed herein but are to understood by the claims and the embodiments covered by the claims and which include, but are not limited to:

1. An ultrasound system comprising;
a handheld ultrasound imaging device including;
a flat two-dimensional (2D) array of micromachined ultrasound transducers (MUTs) having an elevational direction and an azimuthal direction, and
a processor configured to control the 2D array to take a series of ultrasound images along an elevational direction of the 2D array to have successive images taken at a successively progressing angle relative to an axis parallel to the elevational direction of the array by beam steering ultrasonic signals produced by the MUTs, and
a handheld computing device coupled to the handheld ultrasound imaging device and having a memory capable of storing ultrasound image data, and configured to receive ultrasound image data from the handheld ultrasound imaging device and store the ultrasound image data as image data files in the memory, and
including an image processor configured to analyze the ultrasound image data files stored in memory to designate an image from the series of ultrasound images as a key frame and to generate a forward and reversing cine centered on the key frame and that presents a selected series of the stored image data files.

2. The system of claim 1 wherein the image processor is configured to identify a key frame from the selected series of stored image data files which presents within the cine a preferential view of an anatomical target.

3. The system of claim 1 wherein the handheld computing device is configured to display the cine and allow the user to manually pause, fast forward, and rewind the displayed cine.

4. The system of claim 1 wherein the handheld computing device is configured to allow the user to manually select a different image data file in the series of images data files as the key frame.

5. The system of claim 2 wherein the image processor identifies a key frame from the series of image data files by analyzing how clearly a target organ is displayed relative to other ultrasound images of the stored image data files.

6. The system of claim 2 wherein the image processor identifies a key frame from the series of image data files by analyzing visibility of a pathology within an imaging target relative to other images data files of the stored image data files.

7. The system of claim 2 wherein the image processor identifies a key frame from the series of image data files by analyzing how prominently a b-line is displayed in the image data file relative to other image data files of the stored image data files.

8. A method of displaying ultrasound images comprising;
providing a handheld ultrasound imaging device including a flat two-dimensional (2D) array of micromachined ultrasound transducers (MUTs), and
a processor to control the 2D array,
operating the 2D array to take a series of ultrasound images along an elevational direction of the 2D array where each image is taken at a different angle relative to an axis parallel to the elevational direction of the array by beam steering ultrasonic signals produced by the MUTs, and
storing the series of ultrasound images in a memory as a series of ultrasound image data, and
providing a handheld computing device for coupling to the handheld ultrasound imaging device and having a memory capable of storing ultrasound image data, and
transmitting ultrasound image data to the handheld computing device and storing the data in the memory,
image processing the ultrasound images to designate an image from the series of ultrasound images as a key frame, and
displaying ultrasound image data to the user as a cine centered on the key frame.

9. The method of claim 8 wherein the image processor identifies a key frame from the series of ultrasound images which presents within the cine a preferential view of an anatomical target.

10. The method of claim 8 wherein the handheld computing device allows the user to manually pause, fast forward, and rewind a displayed video or cine.

11. The method of claim 8 wherein the handheld computing device allows the user to manually select a different image in the series of ultrasound images as the key frame.

12. The method of claim 9 wherein the image processor identifies a key frame from the series of ultrasound images as displaying a preferential view of an anatomical target by analyzing how clearly a target organ is displayed in the image relative to other images in the series of images.

13. The method of claim 9 wherein the image processor identifies a key frame from the series of ultrasound images as displaying a preferential view of an anatomical target by analyzing visibility of a pathology within an imaging target relative to other images in the series of images.

14. The method of claim 9 wherein the image processor identifies a key frame from the series of ultrasound images as displaying a preferential view of an anatomical target by analyzing how prominently a b-line is displayed in the image relative to other images in the series of images.
